(19) United States
(12) Patent Application Publication
(10) Pub. No.: US 2019/0307527 A1
(43) Pub. Date: Oct. 10, 2019

(54) Fiber Tip Protection Integration For Cannula

(71) Applicant: Alcon Inc., Fribourg (CH)

(72) Inventor: Reto Grueebler, Greifensee (CH)

(21) Appl. No.: 16/373,269

(22) Filed: Apr. 2, 2019

Related U.S. Application Data
(60) Provisional application No. 62/654,953, filed on Apr. 9, 2018.

(51) Int. Cl. A61B 90/30; A61B 17/34

(52) U.S. Cl. CPC A61B 90/30; A61B 2017/00907; A61B 2090/306; A61B 17/3423

(57) ABSTRACT

Illumination of an interior portion of an eye is disclosed herein. In an exemplary aspect, the present disclosure is directed to a cannula assembly. The cannula assembly may include a cannula comprising an outer cannula surface. The cannula assembly may include a cannula hub at a proximal end of the cannula. The cannula assembly may include an optical fiber extending longitudinally along the outer cannula surface, wherein the optical fiber has an optical fiber tip at a distal end of the cannula. The cannula assembly may include a transparent covering disposed over the optical fiber tip.

### CROSS-REFERENCE TO RELATED APPLICATIONS

This application claims the benefit of priority of U.S. Provisional Patent Application Ser. No. 62/654,953 titled “FIBER TIP PROTECTION INTEGRATION FOR CANNULA”, filed on Apr. 9, 2018, whose inventor is Reto Grueebler, which is hereby incorporated by reference in its entirety as though fully and completely set forth herein.

### BACKGROUND

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, and anterior segment procedures, such as cataract surgery. Vitreoretinal surgery may address many different eye conditions, including, but not limited to, macular degeneration, diabetic retinopathy, diabetic vitreous hemorrhage, macular hole, detached retina, epiretinal membrane, and cytomegalovirus retinitis.

During ophthalmic posterior segment surgery, the surgeon may successively use different hand pieces or instruments. A surgical procedure may require that these instruments be inserted into and removed from an incision. Repeated removal and insertion of instruments may cause trauma to the eye at the incision site. To reduce such trauma and allow repeated access to the incision site, hubbed cannulas have been developed and used to help protect the incision site. These devices may include a narrow tube with an attached hub. The tube may be inserted into an incision in the eye up to the hub, which may act as a stop to limit an amount by which the tube from enters the eye. The hub may be stitched to the eye to prevent inadvertent removal.

To visualize the posterior segment of the eye, illumination may be needed in the interior of the eye. For example, the surgeon may need to insert and position a light source to illuminate an interior region of the eye, while simultaneously inserting and positioning a surgical hand piece for cutting and aspirating tissue from the illuminated region.

### SUMMARY

In an exemplary aspect, the present disclosure is directed to a cannula assembly. The cannula assembly may include a cannula having an outer cannula surface; a cannula hub at a proximal end of the cannula; and an optical fiber extending longitudinally along the outer cannula surface. The optical fiber may include an optical fiber distal tip disposed at a distal end of the cannula. The cannula assembly may include a transparent covering disposed over the optical fiber distal tip.

In another exemplary aspect, the present disclosure is directed to a system that may include a light source and a cannula assembly. The cannula assembly may include a cannula that includes an outer cannula surface; a cannula hub disposed at a proximal end of the cannula; an optical fiber coupled to the light source and operable to receive light from the light source. The optical fiber may extend longitudinally along the outer cannula surface. The optical fiber may have an optical fiber distal tip disposed at a distal end of the cannula. The cannula assembly may further include a transparent covering disposed over the optical fiber distal tip.

The different aspects may include one or more of the following features. The cannula may have a length in a range of about 3 millimeters to about 7 millimeters. The cannula may have a cross-sectional shape having an outer radius in a range of about 0.2 millimeters to about 1 millimeter. The cannula hub may have a maximum outer diameter that is larger than two times an outer diameter of the cannula. The cannula hub may have an outer peripheral surface; an arm extension that extends from the outer peripheral surface; and a groove formed in the arm extension. The optical fiber may be disposed in and extend along the groove. The cannula assembly may further include a protective sheath that extends longitudinally along the cannula from the proximal end of the cannula to the distal end of the cannula. The protective sheath may at least partially cover the optical fiber along the cannula. The protective sheath may have an open distal end that allows light to be transmitted from the optical fiber distal tip. The transparent covering may have a light transmission percentage of at least about 90%. The transparent covering may have a refractive index of from about 1 to about 2. The transparent covering may include at least one material selected from the group consisting of an optical adhesive and a thermoplastic polymer. The cannula assembly may further include a transparent sheath disposed over the outer cannula surface around at least a portion of the cannula. The optical fiber may be encased in the transparent sheath. The transparent sheath may include the transparent covering disposed over the optical fiber tip. The cannula assembly may further include a transparent sheath encasing the optical fiber. The transparent sheath and the optical fiber may be disposed in a groove formed in the outer cannula surface. The transparent sheath may include the transparent covering disposed over the optical fiber tip. The transparent sheath may include a fluorinated ethylene propylene copolymer. The transparent covering may include an optical adhesive disposed over the optical fiber distal tip. A surface texture may be disposed on the outer cannula surface at the optical fiber distal tip. The surface texture may be adapted to shape the transparent covering. The cannula may further include an optical fiber guide formed in and extending longitudinally along the outer cannula surface. The optical fiber encased in the transparent sheath may be at least partially disposed in the optical fiber guide. The optical fiber guide may include a reservoir adapted to receive the optical adhesive; and an enlarged end disposed adjacent to the distal end of the cannula. The optical fiber distal tip may be disposed within the enlarged end, and the optical fiber guide may include a surface texture formed in the enlarged portion. The surface texture may be adapted to shape the transparent covering.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

These drawings illustrate certain aspects of some of the embodiments of the present disclosure and should not be used to limit or define the disclosure.

FIG. 1 illustrates a surgical system that includes a cannula with illumination.

FIG. 2 illustrates a longitudinal cross-sectional view of an example cannula disposed in an eye.

FIG. 3A illustrates a longitudinal cross-sectional view of the distal end of an example cannula with a transparent covering on the optical fiber tip.

FIG. 3B illustrates a lateral cross-sectional view of the distal end of an example cannula taken along line 3B-3B of FIG. 3A.

FIG. 4A illustrates a longitudinal cross-sectional view of the distal end of another example cannula with a transparent covering on the optical fiber tip.

FIG. 4B illustrates a lateral cross-sectional view of the distal end of an example cannula taken along line 4B-4B of FIG. 4A.

FIG. 5 illustrates a longitudinal cross-sectional view of the distal end of an example cannula with a transparent covering of an optical adhesive on the optical fiber tip.

FIG. 6A illustrates a top view of the distal end of an example cannula with surface texturing.

FIG. 6B illustrates a top view of the distal end of the example cannula of FIG. 6A with surface texturing after application of a transparent sheath of optical adhesive to an optical fiber.

FIG. 6C illustrates a longitudinal cross-sectional view of the distal end of the example cannula taken along line 6C-6C of FIG. 6B.

FIG. 7 illustrates an example of the surface texturing on the cannula of FIG. 6A.

FIG. 8 illustrates an example of a longitudinal configuration of a transparent covering, which may be applied to the cannula in the form of a drop of the optical adhesive material.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with reference to one or more implementations may be combined with the features, components, and/or steps described with reference to other implementations of the present disclosure. For simplicity, in some instances the same reference numbers may be used throughout the drawings to refer to the same or like parts.

The embodiments described herein generally relate to eye surgery. However, the scope of the application is not so limiting, and the context of eye surgery is provided merely as an example for describing the subject matter of the present disclosure. Consequently, cannulas with illumination may be applicable to other types of procedures including other medical procedures, such as laparoscopic surgical procedures, and the scope of the present disclosure is intended to encompass these other types of medical procedures or other procedures.

More particularly, the described embodiments generally relate to illumination of the interior of an eye with a cannula. The described embodiments include integrating one or more optical fibers into the cannula. To protect the optical fiber tip during insertion into the eye, a protective covering may be disposed over the optical fiber tip. Suitable protective coverings may include, but are not limited to, an optical adhesive material or thermoplastic polymer. The protective covering should be transparent so that light from the optical fiber may be transmitted through the protective covering and into the eye for illumination therein.

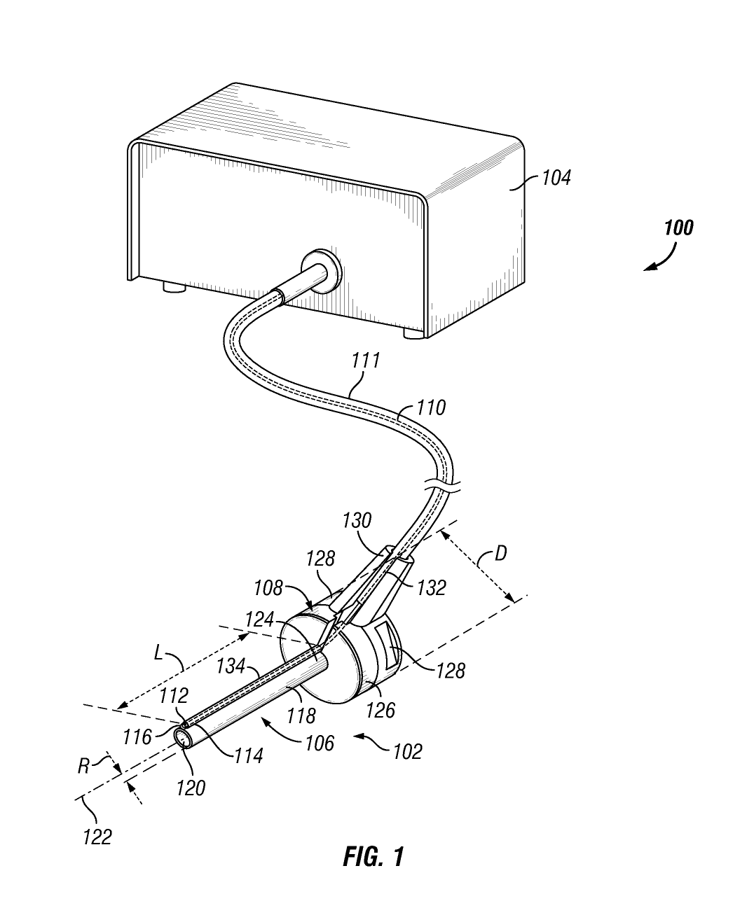

FIG. 1 illustrates an example system 100 that includes a cannula assembly 102 and light source 104. As illustrated, the cannula assembly 102 includes a cannula 106, a cannula hub 108, and an optical fiber 110 operable to conduct light from the light source 104. Light from light source 104 is conducted through optical fiber 110 for emission from optical fiber distal tip 112 at distal end 114 of cannula 106. A transparent covering 116 is disposed over the optical fiber distal tip 112 for protection of the optical fiber distal tip 112.

The cannula 106 includes an outer cannula surface 118 and an inner cylindrical bore 120 that defines a longitudinal axis 122. The cannula 106 has a length L measured parallel to the longitudinal axis 122. In some embodiments, the length L may be in a range of from about 3 millimeters to about 7 millimeters. The cannula 106 also includes an outer radius R. In some embodiments, the outer radius R may be in range of from about 0.2 millimeters to about 1 millimeters. However, the scope of the disclosure is not so limited to these values of length L and outer radius R. Rather, the cannula 106 may have any dimensions as desired or needed for a particular application.

As illustrated, the cannula hub 108 adjoins a proximal end 124 of the cannula 106. In some embodiments, a maximum outer diameter D of the cannula hub 108 may be larger than two times an outer diameter of the cannula 106, where the outer diameter of the cannula 106 is two times the outer radius R. In some embodiments, the outer peripheral surface 126 of the cannula hub 108 may include at least two gripping flats 128, for example, to facilitate gripping by a surgeon with tweezers (not shown) or other gripping device. In some instances, an arm extension 130 may extend from the outer peripheral surface 126 of the cannula hub 108. The arm extension 130 includes a groove 132 in which the optical fiber 110 may be disposed. The groove 132 extends through the arm extension 130 and into the cannula hub 108. The optical fiber 110 extends through the groove 132 and along the outer cannula surface 118 of the cannula 106.

With continued reference to FIG. 1, the cannula assembly 102 further includes a protective sheath 134. The protective sheath 134 is coupled to the outer cannula surface 118 of the cannula 106 and extends longitudinally along the cannula 106 from the proximal end 124 to the distal end 114. The optical fiber 110 is attached to and extends along the cannula 106. For example, in some instances, the optical fiber 110 may extends along the entire length L of the cannula 106. In other instances, the optical fiber 110 may extend along only a portion of the length L of the cannula 106. For example, in some instances, where the optical fiber 110 extends along less than the entire length L, the optical fiber distal tip 112 is proximal to a distal tip of the cannula 106. The protective sheath 134 covers the optical fiber 110 for all or a portion of a length that the optical fiber 110 extends along the cannula 106. The protective sheath 134 may be made from any suitable or useful material for protecting the optical fiber 110. Example materials from which the protective sheath 134 may be formed include, but are not limited to, metals and plastics, among others. To allow light transmission from the optical fiber 110, the protective sheath 134 may be open at the distal end 114 of the cannula 106. In some instances, the protective sheath 134 may be opaque so that light traveling through the optical fiber 110 is prevented from being emitted from anywhere along the length of the optical fiber 110 except from the optical fiber distal tip 112 at dial end 114 of cannula 106. In some embodiments, the protective sheath 134 may be transparent and may include the transparent covering 116. Thus, in some implementations, the protective sheath 134 may be transparent, and the transparent covering 116 may be incorporated into the protective sheath 134 as an integral part.

The optical fiber 110 may have any of a variety of configurations. In some embodiments, the optical fiber 110 may be a glass optical fiber. However, embodiments are not so limited. Rather, the optical fiber 110 may include other suitable materials for light transmission, including, but not limited to, plastics and glass, as may be desired for a particular application. In some embodiments (not shown), the optical fiber 110 may be a strand of optical fibers. In some embodiments (not shown), the optical fiber 110 between optical fiber distal tip 112 and light source 104 may include two more optical fibers coupled together, e.g., coupled in an end to end arrangement, for example. As illustrated, the optical fiber 110 includes an outer cladding 111 or other protective layer or layers along at least a portion of the length of the optical fiber 110. In the illustrated example, the outer cladding 111 disposed over the optical fiber 110 extends from the cannula assembly 102 to the light source 104.

The light transmitted by the optical fiber 110 may be emitted from optical fiber distal tip 112. The light may be generated remotely from the optical fiber 110. For example, in the system 100 shown in FIG. 1, the optical fiber 110 is coupled to the light source 104 that is remote from the cannula assembly 102. In some embodiments, the light source 104 to which the optical fiber 110 is coupled may be provided in a surgical console (not shown). The light source 104 may include any of a variety of different types of light source for generating light for delivery through the optical fiber 110. For example, light sources for inclusion in the light source 104 may include, but are not limited to, one or more of a light-emitting diode (LED) light source, a phosphor light source, or a laser light source. Non-limiting examples of laser light sources include monochromatic (e.g., infrared, visible), multi-spectral, or supercontinuum white lasers.

As shown in FIG. 1, the optical fiber distal tip 112 is covered by the transparent covering 116. The transparent covering 116 protects the optical fiber distal tip 112 that would otherwise be exposed at the distal end 114 of the cannula 106. Transparency is the ability of a material to transmit light. As disclosed herein, a material is considered transparent where it has light transmission percentage of over 85% as measured using ASTM D-1003. The transparent covering 116 is characterized as being transparent with a light transmission percentage of over 85% so that light from the optical fiber distal tip 112 may be transmitted through the transparent covering 116. In some embodiments, the transparent covering 116 may have a light transmission percentage of at least about 90%, at least about 95%, at least about 98%, or about least about 99%.

In addition to transparency, the refractive index of the transparent covering 116 may also determine the ability of the transparent covering 116 to transmit light. The refractive index (also referred to as index of fraction) is a dimensionless number that describes how light propagates through a material. The refractive index is defined as the ratio of the speed of light in a vacuum to that in a specified matter. The refractive index for the transparent covering 116 is the ratio of the phase velocity of light in a vacuum to the phase velocity of light in the transparent covering 116. In some embodiments, the transparent covering 116 may have a refractive index of from about 1 to about 2. However, the scope of the disclosure is not so limited to these values of refractive index. Rather, the transparent covering 116 may have any refractive index as desired for a particular application. In some embodiments, the refractive index of the transparent covering 116 may be selected to substantially the same as an infusion fluid used during the course of a surgical procedure. As used herein, the refractive index of the transparent covering 116 and the infusion fluid (e.g., saline) are substantially the same where there is no more than a 10% variance between the respective indices. By having the refractive indices substantially the same as the infusion fluid, the illumination pattern from the optical fiber distal tip 112 may be more evenly distributed.

Examples of the transparent covering 116 may include any transparent material that can protect the optical fiber distal tip 112 while allowing light to pass therethrough. Example materials from which the transparent covering 116 may be made include, but are not limited to, optical adhesives and thermoplastic polymers. It should be understood that the transparency of the materials forming the transparent covering 116 may depend on a number of factors, including, but not limited to, a thickness of the transparent covering 116. Examples of optical adhesives include, but are not limited to, acrylic-based adhesives. Examples of thermoplastic polymers include, but are not limited to, thermoplastic fluoropolymers, such as fluorinated ethylene propylene copolymers (FEP) and acrylate-based polymers. The transparency and refractive index of a material selected for the transparent covering 116 may be selected based on a particular application for which the cannula assembly 102 is to be used.

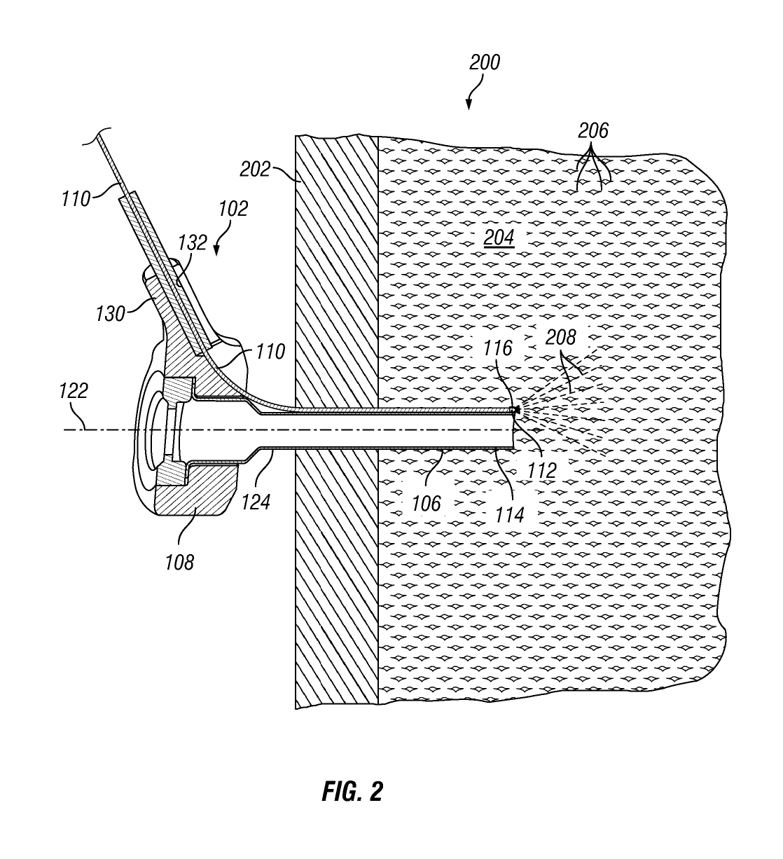

FIG. 2 illustrates a longitudinal cross-sectional view of the example cannula assembly 102 disposed in an eye 200. As illustrated, the cannula assembly 102 includes the cannula 106 and the cannula hub 108. The cannula hub 108 adjoins the proximal end 124 of the cannula 106. The cannula hub 108 is positioned outside the eye 200. The cannula hub 108 may be located proximate to, or in contact, with sclera 202 of the eye 200. In the illustrated example, the cannula 106 is inserted through the sclera 202 into an interior portion 204 of the eye 200. The transparent covering 116 is disposed over the optical fiber distal tip 112 at the distal end 114 of the cannula 106 to protect the optical fiber distal tip 112 and prevent damage to the optical fiber 110 during insertion of the cannula 106 into the eye 200, e.g., through the sclera 202 and a material 206 (e.g., vitreous humor or infusion fluid) present within the eye 200. While not shown, a trocar or other instrument may be used with the cannula assembly 102 to pierce the sclera 202. The optical fiber 110 extends through the groove 132 in the arm extension 130 of the cannula hub 108 and along the cannula 106, for example, in parallel to the longitudinal axis 122 of the cannula 106. Although a protective sheath 134 described in the context of the example shown in FIG. 1 is not included in the example illustrated in FIG. 2, a protective sheath 134 may be included.

The optical fiber distal tip 112 of the optical fiber 110 is disposed at the distal end 114 of the cannula 106. The optical fiber 110 transmits light from a light source (e.g., light source 104 shown on FIG. 1) to the optical fiber distal tip 112 where the light is emitted as a light beam 208 into the interior portion 204 of eye 200. The light beam 208 emitted from the optical fiber distal tip 112 is transmitted through the transparent covering 116 and into the interior portion 204. In this manner, the optical fiber 110 is operable to provide illumination into the interior portion 204 of the eye 200 to facilitate visualization in the interior portion 204 while maintaining protection of the optical fiber distal tip 112 by the transparent covering 116, such as during insertion of the cannula 106 into the eye 200.

Any of a variety of different techniques may be used for application of the transparent covering 116 to the optical fiber distal tip 112, and one technique may be selected over another depending on, for example, a particular material selected for the transparent covering 116. For example, thermoplastic fluoropolymers (e.g., FEP) may be applied to the optical fiber distal tip 112 by a number of different techniques, including, but not limited to, heat shrinking, foil wrapping, dip coating, or foil wrapping. In addition, techniques may rely on capillary technique in coating the entire optical fiber 110 or only the optical fiber distal tip 112. In addition, embodiments may include covering the optical fiber distal tip 112 with an optical adhesive. An optical adhesive may be applied in the form of one or more drops directed onto optical fiber 110 proximate to the distal end 114 of the cannula 106. The optical adhesive forms the transparent covering 116 and serves both to protect the optical fiber distal tip 112 and to form a transparent pathway for the transmission of light emitted from the optical fiber distal tip 112.

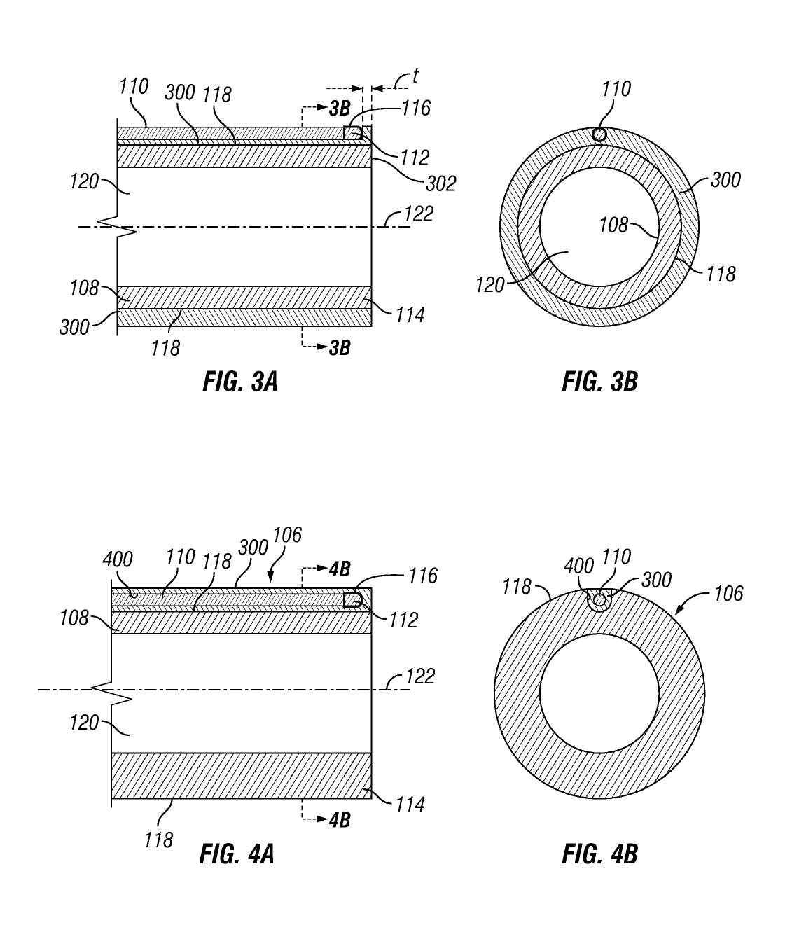

FIG. 3A illustrates a longitudinal cross-sectional view of a distal end 114 of an example cannula 106. The cannula 106 defines an inner cylindrical bore 120 that defines a longitudinal axis 122, and an optical fiber 110 is coupled to an outer surface 118 of the cannula 106. A transparent covering 116 is disposed over an optical fiber 110. FIG. 3A shows the optical fiber 110 embedded in a transparent sheath 300 and that the transparent covering 116 disposed over and protecting the optical fiber 110. Although the transparent covering 116 is shown as a separate element, the transparent material that forms the transparent sheath 300 also forms the transparent covering 116. Thus, the transparent sheath 300 and the transparent covering 116 form a continuous covering of a protective material applied to the optical fiber 110. In other instances, the protective material forming the transparent covering 116 may be limited to covering only the optical fiber distal tip 112 while the remainder of the optical fiber 110 extending along the cannula 106 may be covered by a thin-walled metal or plastic tube to protect the optical fiber 110. The transparent sheath 300 may be formed of a transparent polymer, such as, but not limited to, thermoplastic fluoropolymers, such as FEP and acrylic-based polymers. The transparent sheath 300 may be disposed over outer cannula surface 118 around at least a portion of the cannula 106. In the illustrated example, the transparent sheath 300 adheres the optical fiber 110 to the outer cannula surface 118. The optical fiber 110 extends along the cannula 106 parallel to the longitudinal axis 122 of the cannula 106. As illustrated, the transparent sheath 300 includes the transparent covering 116 disposed over the optical fiber distal tip 112. The optical fiber distal tip 112 may be aligned with distal tip 302 of the cannula 106 or may be proximal thereto, as shown on FIG. 3A. In some instances, the optical fiber distal tip 112 may be displaced proximally from the distal end 302 of the cannula 106 in a range from about 0 millimeters to about 5 millimeters.

FIG. 3B illustrates a transverse cross-sectional view of the distal end 114 of the example cannula 106 of FIG. 3A taken along line 3B-3B. As illustrated, the optical fiber 110 is embedded in a transparent sheath 300. The transparent sheath 300 is disposed over an entire circumference of the outer cannula surface 118 along the entire length L of the cannula 106 (as illustrated in FIG. 1, for example). In other instances, the transparent sheath 300 around an entire circumference of the outer surface 118 but along only a portion of a length of the optical fiber 110 that extends along the length L of the cannula 106. In still other instances, the transparent sheath 300 may be formed around less than an entire circumference of the outer cannula surface 118 and along all or only a portion of the length of the optical fiber 110 that extends along the length L of the cannula 106. In the illustrated example of FIG. 3B, the entire circumference of the outer cannula surface 118 at the distal end 114 is covered by the transparent sheath 300. In the illustrated example, the transparent sheath 300 adheres the optical fiber 110 to the outer cannula surface 118.

FIG. 4A illustrates a longitudinal cross-sectional view of the distal end 114 of another example of the cannula 106. The cannula 106 defines an inner cylindrical bore 120, and the cylindrical bore 120 defines a longitudinal axis 122. An optical fiber 110 extends along a length of L of the cannula 106 (as illustrated in FIG. 1, for example), and a transparent covering 116 is disposed over the optical fiber 110. In the illustrated example, the portion or portions of the optical fiber 110 in contact with the transparent sheath 300 is/are embedded in a transparent sheath 300. As shown in FIG. 4A, the portion or portions of the optical fiber 110 in contact with the transparent sheath 300 is/are fully encased or fully immersed within the material forming the transparent sheath 300. The transparent sheath 300 and transparent covering 116 may be formed of a transparent polymer. The transparent polymer may be formed from materials including, include, but not limited to, thermoplastic fluoropolymers, such as FEP and acrylic-based polymers. In contrast to the example shown in FIGS. 3A and 3B, the transparent sheath 300 shown in FIG. 4A coats the optical fiber 110, but does not fully cover the entire circumference of the outer cannula surface 118. Rather, as shown in FIG. 4B, the transparent sheath 300, the optical fiber 110, and the transparent covering 116 are disposed within a groove 400 formed within the cannula 106. The groove 400 is formed in the outer cannula surface 118. In the illustrated example, the groove 400 extends along the cannula 106 parallel to the longitudinal axis 122, and the optical fiber 110 is shown also extending through the groove 400 in a manner parallel to the longitudinal axis 122, although the scope of the disclosure is not so limited. That is, in some implementations, one or both of the groove and the optical fiber 110 may extends through the cannula in a manner that is not parallel to the longitudinal axis 122. As illustrated, the material forming the transparent sheath 300 is also disposed over the optical fiber distal tip 112 to form the transparent covering 116. Again, though, in other implementations, the transparent, protective material may be applied to cover the optical fiber distal tip 112 (thus, forming the transparent covering 116) and the remainder of the optical fiber 110 extending along the cannula 106 may be covered by a sheath formed from a different material, e.g., a plastic or metal.

FIG. 4B illustrates a transverse cross-sectional view of the distal end 114 of the example cannula 106 of FIG. 4A taken along line 4B-4B. As illustrated, the optical fiber 110 is embedded in a transparent sheath 300, and both the optical fiber 110 and the transparent sheath 300 are disposed in a groove 400 formed in the cannula 106. As a result, the transparent sheath 300 forms a portion of the outer cannula surface 118. As shown, the optical fiber 110 is fully immersed in the transparent sheath 300.

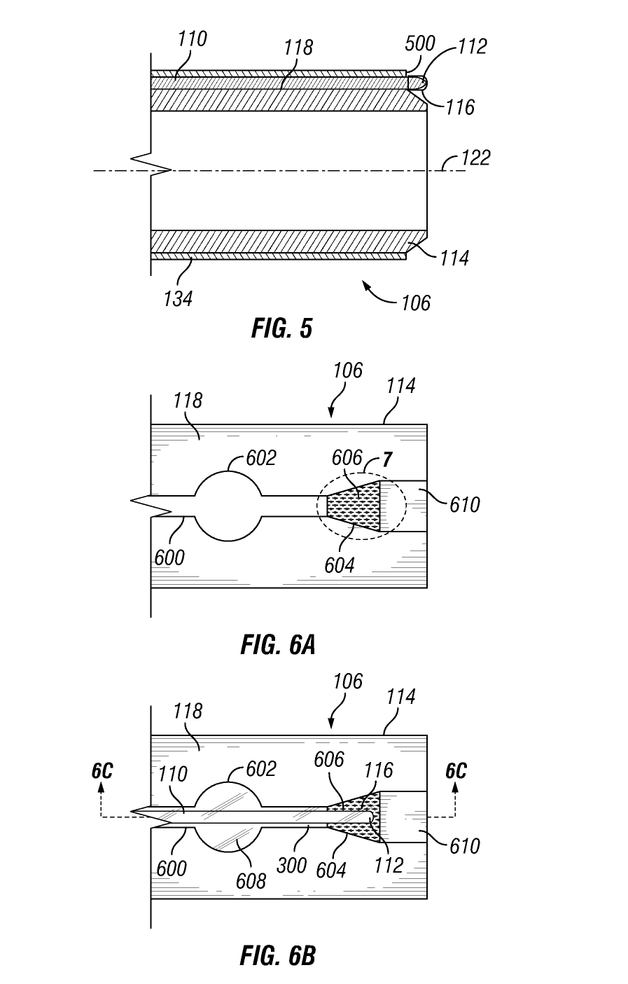

FIG. 5 illustrates a longitudinal cross-sectional view of the distal end 114 of an example cannula 106 with a transparent covering 116 formed on the optical fiber distal tip 112. The transparent covering 116 may be applied by coating the optical fiber distal tip 112 with an optical adhesive. A transparent sheath 134 may be formed from either the same material forming the transparent covering 116 or a different material, e.g., a plastic, metal, or other material different from that forming the transparent covering 116. In some implementations, a transparent adhesive may be applied to form both the transparent sheath 300 and the transparent covering 116. As illustrated, the optical fiber 110 is coupled to the outer cannula surface 118. The optical fiber 110 extends along the cannula 106, for example, parallel to the longitudinal axis 122 of the cannula 106. A protective sheath 134 surrounds the entire circumference of the outer cannula surface 118, sandwiching the optical fiber 110 between the protective sheath 134 and the outer cannula surface 118, thereby securing the optical fiber 110 to the cannula 106. The protective sheath 134 may cover the optical fiber 110 for all or portion of the span of the optical fiber 110 along the cannula 106.

In the illustrated example, the distal end 114 of the cannula is chamfered, and the optical fiber distal tip 112 extends beyond the distal tip 500 of the protective sheath 134. In some implementations, the optical fiber 110 may be positioned on the cannula 106 such that the chamfered or inwardly tapered surface reduces or eliminates the generation of shadowing caused by the cannula 106, thereby improving the quality of illumination provided by the cannula assembly. The transparent covering 116 is formed over the optical fiber distal tip 112. As previously described, the transparent covering 116 may be in the form of an optical adhesive applied to the optical fiber distal tip 112 so as to coat an entirety of the optical fiber distal tip 112. An optical adhesive may be applied to the optical fiber distal tip 112, for example, by dip coating and drip coating.

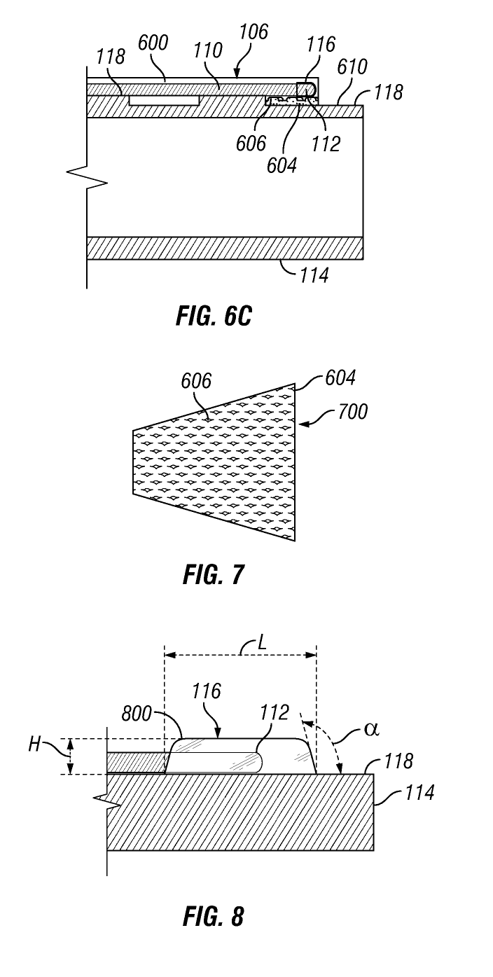

Referring to FIGS. 2 and 5, application of an optical adhesive to the optical fiber distal tip 112 to form the transparent covering 116 may affect the illumination quality of light transmitted from the optical fiber 110. For example, differences in the refractive indices of the transparent covering 116 and the filler material 206 within the interior portion 204 of the eye 200 may result in undesirable refraction of the light beam 208 as it passes through the transparent covering 116 such that illumination in the interior portion 204 of the eye 200 is inconsistent or otherwise undesirable. To lessen an amount of refraction introduced by the transparent covering 116, the transparent covering 116 may be shaped. Example techniques for shaping the transparent covering 116 of the optical adhesive will be described below with respect to FIGS. 6A and 6B.

FIGS. 6A to 6C illustrate an example technique for shaping the transparent covering 116 formed from an optical adhesive. With reference now to FIG. 6A, FIG. 6A illustrates a top view of a distal end 114 of a cannula 106. In the illustrated example, the cannula 106 includes an outer cannula surface 118 that includes an optical fiber guide 600. The optical fiber guide 600 is in the form of a groove that extends longitudinally along the outer cannula surface 118 and defines a reservoir 602 adapted to receive an optical adhesive. The optical fiber guide 600 further includes an enlarged end 604 that tapers outwardly in the distal direction. As illustrated, the enlarged end 604 of the optical fiber guide 600 includes a surface texture 606. By including the surface texture 606 onto the enlarged end 604, a wettability (i.e., the ability of a fluid to spread on or adhere to a surface) of the enlarged end 604 to the optical adhesive is controlled. The surface texture 606 may be arranged, for example, to provide a geometric guide for the optical adhesive. The geometric guide controls flow, for example, of a liquid material applied to the optical fiber 110 that will ultimately form the transparent covering 116 once cured. The geometric guide controls both the flow of the applied liquid material (e.g., in the form of one or more drops) and ultimately controls or at least partially contributes to the resultant shape of the transparent covering 116. Thus, the geometric guide affects the wettability of the enlarged end 604 to control the shape of the applied material that will ultimately form the transparent covering 116 (formed, for example, from an adhesive drop) and, therefore, to control the resulting shape of the transparent covering 116. The wettability of the enlarged area 604 may also be used to control a contact area formed between the transparent covering 116 and the outer cannula surface 118.

The surface texture 606 may be arranged in a structured pattern to influence the shape of the transparent covering 116. For example, the surface texture 606 may be arranged on the enlarged end 604 in a diamond pattern 700, as shown on FIG. 7. The diamond pattern 700, as well as other surface textures, are operable to control a shape of the transparent covering 116 as the optical adhesive cures. It should be understood, however, that the configuration of the surface texture 606, including geometry, structure, and distribution of asperities (i.e., the surface features that collectively form the surface texture 606) may vary. For example, the surface texture 606 may be regular and symmetric. In other instances, the surface texture 606 may be irregular and asymmetric. The surface texture 606 may be applied to the enlarged end 604 in numerous ways. For example, applicable techniques include, but are not limited to, grinding, laser ablation, and electrochemical machining, among others. Referring to FIG. 6C, a distal end portion 610 of the outer cannula surface 118 is recessed. By recessing the distal end portion 610, obstruction by distal end 114 of the cannula 106 of the light emitted from the optical fiber distal tip 112 may be reduced, thereby reducing the generation of shadowing within an area being illuminated by the cannula assembly.

FIGS. 6B and 6C are top and longitudinal cross-sectional views, respectively, of the distal end 114 of the cannula 106 shown in FIG. 6A. The cross-sectional view of FIG. 6C is taken along line 6C-6C, as shown in of FIG. 6B. An optical fiber 110 is disposed in the optical fiber guide 600.

Referring to FIG. 6B, an optical adhesive 608 is shown as having been introduced into reservoir 602 of the optical fiber guide 600. A volume of the optical adhesive 608 that is introduced into the reservoir 602 may be controlled so that the optical adhesive 608 flows out of the reservoir 608 along the optical fiber guide 600 such that the optical adhesive encases at least a portion of the optical fiber 110 in the form of a transparent sheath 300 within the optical fiber guide 600. As illustrated, the transparent sheath 300 includes a transparent covering 116 at optical fiber distal tip 112. The transparent covering 116 is disposed over the optical fiber distal tip 112 to provide protection thereto, and the transparent covering 116 may be formed in a shape adapted to provide uniform illumination to a surgical field. Further, the shape of the transparent covering 116 may be configured such that the transmitted light from the optical fiber distal tip 112 has a defined angular spread. Example angles of this optical spread are described in more detail below. In the illustrated example, the enlarged end 604 of the optical fiber guide 600 includes surface texturing 606.

By applying a selected amount of the optical adhesive 608 to the reservoir 602, the transparent covering 116 takes the form of an elongated drop 800, as shown on FIG. 8. As illustrated on FIG. 8, the elongated drop 800 is disposed over at least a portion of optical fiber 110, including optical fiber distal tip 112. The transparent covering 116 may be considered elongated as it may have a length L that is greater than its height H. For example, in some instances, the length L of the transparent covering 116 may be more than double the height H. With continued reference to FIG. 8, the transparent covering 116 defines a contact angle α with outer cannula surface 118 of cannula 114. In some instances, the contact angle α may be within a range of about 90° to about 150°. However, other angles α are contemplated. For example, in some instances, the angle α may be greater than 150° or less than 90°. In this manner, the enlarged end 604 and the surface texture 606 formed thereon (e.g., shown on FIGS. 6C and 7) are used to control the shape of the transparent covering 116 along a portion of the transparent covering 116 adjacent to the enlarged end 604 of the optical fiber guide 600. Particularly, in this portion of the transparent covering 116, the transparent covering 116 has a tapered shape that increases in the distal direction (as shown in FIGS. 6A, 6B, and 7). In addition, a surface texture of the transparent covering 116 that is in contact with the surface texture 606 is the negative of the surface texture 606. By controlling the shape of the transparent covering 116, the refraction of the light beam 208 (e.g., FIG. 2) is controlled.

It is believed that the operation and construction of the present disclosure will be apparent from the foregoing description. While the various example apparatuses and methods are described above, the scope of the present disclosure encompasses various changes and modifications may be made thereto without departing from the spirit and scope of the disclosure as defined in the following claims.

What is claimed is:

1. A cannula assembly comprising:
a cannula comprising an outer cannula surface;
a cannula hub disposed at a proximal end of the cannula;
an optical fiber extending longitudinally along the outer cannula surface, the optical fiber comprising an optical fiber distal tip disposed at a distal end of the cannula; and
a transparent covering disposed over the optical fiber distal tip.

2. The cannula assembly of claim 1, wherein the cannula further comprises a length and a cross-sectional shape having an outer radius, wherein the length of the cannula is in a range of about 3 millimeters to about 7 millimeters, and wherein the outer radius of the cross-sectional shape in a range of about 0.2 millimeters to about 1 millimeter.

3. The cannula assembly of claim 1, wherein a maximum outer diameter of the cannula hub is larger than two times an outer diameter of the cannula.

4. The cannula assembly of claim 1, wherein the cannula hub comprises:
an outer peripheral surface;
an arm extension that extends from the outer peripheral surface; and
a groove formed in the arm extension, and
wherein the optical fiber is disposed in and extends along the groove.

5. The cannula assembly of claim 1, further comprising a protective sheath that extends longitudinally along the cannula from the proximal end of the cannula to the distal end of the cannula,
wherein the protective sheath at least partially covers a portion of the optical fiber that extends along the cannula, and
wherein the protective sheath has an open distal end that allows light to be transmitted from the optical fiber distal tip.

6. The cannula assembly of claim 1, wherein the transparent covering has a light transmission percentage of at least about 90%.

7. The cannula assembly of claim 1, wherein the transparent covering has a refractive index of from about 1 to about 2.

8. The cannula assembly of claim 1, wherein the transparent covering comprises at least one material selected from the group consisting of an optical adhesive and a thermoplastic polymer.

9. The cannula assembly of claim 1, further comprising a transparent sheath disposed over the outer cannula surface around at least a portion of the cannula, wherein the optical fiber is encased in the transparent sheath, and wherein the transparent sheath comprises the transparent covering disposed over the optical fiber distal tip.

10. The cannula assembly of claim 1, further comprising a transparent sheath encasing the optical fiber, wherein the transparent sheath and the optical fiber are disposed in a groove formed in the outer cannula surface, and wherein the transparent sheath comprises the transparent covering disposed over the optical fiber distal tip.

11. The cannula assembly of claim 10, wherein the transparent sheath comprises a fluorinated ethylene propylene copolymer.

12. The cannula assembly of claim 10, wherein the transparent covering comprises an optical adhesive disposed over the optical fiber distal tip.

13. The cannula assembly of claim 12, wherein a surface texture is disposed on the outer cannula surface at a location adjacent to the optical fiber distal tip, the surface texture adapted to shape the transparent covering.

14. The cannula assembly of claim 12, wherein the cannula further comprises an optical fiber guide formed in the outer cannula surface and extending longitudinally along a length of the cannula, wherein the optical fiber encased in the transparent sheath is at least partially disposed in the optical fiber guide, wherein the optical fiber guide comprises:
a reservoir adapted to receive the optical adhesive; and
an enlarged end disposed adjacent to the distal end of the cannula, wherein the optical fiber distal tip is disposed within the enlarged end, and wherein the optical fiber guide comprises a surface texture formed in the enlarged end, the surface texture adapted to shape the transparent covering.

15. A system comprising:
a light source;
a cannula assembly comprising:
a cannula comprising an outer cannula surface;
a cannula hub disposed at a proximal end of the cannula;
an optical fiber coupled to the light source and operable to receive light from the light source, wherein the optical fiber extends longitudinally along the outer cannula surface, and the optical fiber comprising an optical fiber distal tip disposed at a distal end of the cannula; and
a transparent covering disposed over the optical fiber distal tip.

16. The system of claim 15, wherein a maximum outer diameter of the cannula hub is larger than two times an outer diameter of the cannula, and wherein the cannula hub comprises:
an outer peripheral surface;
an arm extension that extends from the outer peripheral surface; and
a groove formed in the arm extension, wherein the optical fiber is disposed in and extends along the groove.

17. The system of claim 15, wherein the transparent covering comprises an optical adhesive disposed over the optical fiber distal tip.

18. The system of claim 15, wherein the cannula further comprise an optical fiber guide formed in the outer cannula surface and extending longitudinally along a length of the cannula, wherein the optical fiber is encased in a transparent sheath, wherein the optical fiber is at least partially disposed in the optical fiber guide, wherein the optical fiber guide comprises:
a reservoir adapted to receive an optical adhesive; and
an enlarged end disposed adjacent to the distal end of the cannula, wherein the optical fiber tip is disposed in the enlarged end, and wherein the optical fiber guide comprises a surface texture formed in the enlarged end, the surface texture adapted to shape the transparent covering.

19. The system of claim 15, wherein the cannula assembly further comprises a transparent sheath disposed over the outer cannula surface around at least a portion of the cannula, wherein the transparent sheath comprises the transparent covering disposed over the optical fiber tip.

20. The system of claim 15, wherein the cannula assembly further comprises a transparent sheath encasing the optical fiber, wherein the transparent sheath and the optical fiber are disposed in a groove formed in the outer cannula surface, and wherein the transparent sheath comprises the transparent covering disposed over the optical fiber distal tip.
